(19) United States
(12) Patent Application Publication
(10) Pub. No.: US 2018/0110406 A1
(43) Pub. Date: Apr. 26, 2018

(54) THREE DIMENSIONAL VISION SYSTEM FOR INTERVENTIONAL SURGERY

(71) Applicants: Swati SARNAIK, Pune (IN); Suresh PATANKAR, Pune (IN)

(72) Inventors: Swati SARNAIK, Pune (IN); Suresh PATANKAR, Pune (IN)

(21) Appl. No.: 15/568,543

(22) PCT Filed: Jan. 28, 2016
(86) PCT No.: PCT/IB2016/050439

§ 371 Date: Oct. 23, 2017

(30) Foreign Application Priority Data
Apr. 21, 2015 (IN) 1625/MUM/2015

(51) Int. Cl. A61B 1/12; A61B 1/313; A61B 1/00; A61B 1/015; A61B 1/05; A61B 1/06; A61B 90/00

(52) U.S. Cl. CPC A61B 1/126; A61B 1/3132; A61B 1/00087; A61B 1/00091; A61B 1/015; A61B 90/50; A61B 1/0676; A61B 1/0684; A61B 1/00135; A61B 90/361; A61B 1/00149; A61B 1/05

(57) ABSTRACT

Disclosed herein is a self-illuminated and self-cleaning three-dimensional vision system for interventional surgery the actuation of which is based upon triangulation that allows achievement of a 360° rotatable spherical dome view-envelope without interference with other surgical equipment at the site of surgical intervention or requirement of external maneuvers, reiterative calibration and referencing on part of the user.

### CROSS REFERENCES TO RELATED APPLICATIONS

This application is a National stage application from PCT application PCT/IB2016/050439 filed on Jan. 28, 2016, which claims priority to Indian provisional application for patent No. 1625/MUM/2015 Apr. 21, 2015 the contents of which are incorporated herein in their entirety by reference.

### FIELD OF THE INVENTION

The present invention belongs to the field of surgical equipment, and more particularly to the construction and operations of a three-dimensional viewing scope system intended primarily for application in minimally invasive surgery.

### BACKGROUND OF THE INVENTION AND DESCRIPTION OF RELATED ART

In minimally invasive surgery surgical instruments are inserted in the patient's body through small holes. Such technique is aimed at reducing the amount of extraneous tissue that is damaged during diagnostic or surgical procedures, thereby reducing patient recovery time, discomfort, and deleterious side effects.

Minimally invasive surgical procedures including arthroscopy, retroperitoneoscopy, pelviscopy, nephroscopy, cystoscopy, cisternoscopy, sinoscopy, hysteroscopy, urethroscopy generically involve functionalities including clamping, grasping, scissoring, stapling, manipulating cameras/needle holders and the like which demand a high level of dexterity, accuracy and precision. However, the surgery site is not accessible to direct vision of the surgeon and must be viewed indirectly on external displays. Therefore, a provision for real time view is essential for the surgeon concerned. Existing systems are severely limited by smaller view-envelope and greater maneuver envelope which obstruct motion of the surgeon in addition to lacking option of flexible insertion, thus proving ineffective to address needs presented.

Endoscopy is a minimally invasive diagnostic technique where a camera/vision system is needed to be inserted at the area of diagnosis through another small incision on patient's body in order to have a view inside of the site of surgical intervention. However, as mentioned before, the conventional 3D vision systems for minimally invasive surgical techniques are limited in their view envelope and require external manipulation to be able to have wider field of view. Furthermore, dexterity constraints also prevent the optimal placement of the camera for satisfactorily viewing the site of surgery. In general, motion of these systems is highly limited due to such constraints.

Additionally, due to splash of body fluids or condensation frequent cleaning of camera distal end is needed. It is advantageous for the surgery if the procedure is completed without or minimal removal of the camera system avoiding added time in removal and replacement and referencing of the camera.

Prior art lists some attempts for achieving the ideal vision system wanted in the art. For example, US20140180001 discloses an endoscope comprising a system with multiple cameras for use in minimal invasive surgery. This prior art claims multiple cameras inserted separately through a tube. The cameras can be rotated or tilted on the outer surface of the tube. This device has no arrangement for the multiple view points and multiple view angles. Hence the dome view envelope is not possible.

Another reference, U.S. Pat. No. 7,339,341 discloses a surgical camera robot to be placed entirely within an open space such as an abdominal cavity. The instant camera robot has pan and tilt capabilities, an adjustable focus camera, a support component for supporting the robot body and a handle to position the camera. This system has limited view envelope and do not allow dome view. For changing the view envelope an external manipulator is necessary. To obtain pan and tilt the entire cylindrical body enclosing camera needs to be moved. Pan and tilt is difficult in close proximity of organs. Also this could be unsafe to the nearby organs and tissues or the movement of the whole cylindrical body in such proximity. It also has a disadvantage where the handle of the camera system needs to be visible all the time. Placing a camera system directly on the patient's organs or internal walls might create stability issues as there would be a natural movement or vibrations of the human body and organs.

Yet another reference, US20130310648 discloses a 360 degree panning stereo endoscope. It claims two movable cameras that have fixed direction of view angles. In this prior art the plane of camera is fixed hence dynamic change of the view plan is not possible. The stereoscopic cameras are not mounted on the same reference hence relative motion between two cameras cannot be avoided. Also this system is hand operated is prone to vibrations of human handling.

From a concerted learning from the existing state-of-art, there is yet felt a need to overcome the persisting drawbacks and provide a vision system mechanics for 3D and conventional camera system used for endoscopy and minimal invasive surgery to avoiding larger cuts and ensuring lesser trauma to patient, less post-operative pain and faster recovery of the patient which also has inbuilt light source to facilitate illuminated view. No system hitherto available is effective in addressing this need of the art.

Background art, therefore to the limited extent presently surveyed, does not list a single effective solution embracing all considerations mentioned hereinabove, thus preserving an acute necessity-to-invent for the present inventor who, as result of his focused research, has come up with novel solutions for resolving all needs of the art once and for all. Work of the presently named inventor, specifically directed against the technical problems recited hereinabove and currently part of the public domain including earlier filed patent applications, is neither expressly nor impliedly admitted as prior art against the present disclosures.

A better understanding of the objects, advantages, features, properties and relationships of the present invention will be obtained from the following detailed description which sets forth an illustrative yet-preferred embodiment.

### OBJECTIVES OF THE PRESENT INVENTION

The present invention is identified in addressing at least all major deficiencies of art discussed in the foregoing section by effectively addressing the objectives stated under, of which:

It is a primary objective, to provide for the construction and operation of a vision system for interventional surgery which is capable of allowing least interference, rotatable, spherical view-envelope at the site of surgical intervention, or in other words, stereoscopic or 3D imaging of the site of intervention/surgery.

It is another objective of the present invention, in addition to the above objective(s), that the vision system so provided is capable of allowing a user to avail real time stereoscopic view at the site of intervention/surgery in a manner characterized by concerted motion of all camera modules involved without need for reiterative referencing and calibration.

It is another objective of the present invention, in addition to the above objective(s), that the vision system so provided is capable of allowing a wide, flexible, spherical dome view-envelope yet within a minimal maneuvering envelope, which imply smaller incisions and therefore lesser trauma for insertion into the patient's body besides avoiding obstruction to surgical instruments inside or outside of the patient's body.

It is another objective of the present invention, in addition to the above objective(s), that the vision system so provided is augmented with a self-cleaning mechanism so as to avoid obstruction of view by blood and other fluids and also minimizing repeated re-insertion and/or referencing at site of intervention/surgery.

It is another objective of the present invention, in addition to the above objective(s), that the vision system so provided has means to enhance dexterity of the user while minimizing vibrations ensuing in the application environment.

It is another objective of the present invention, in addition to the above objective(s), that the vision system so provided has self-illuminating means that negate insertion of another light source into the patient's body and also ensure same relative light direction even after changing the field of view thereby avoiding further adjustment of the light source with respect to the camera module after changing the field of view.

It is another objective of the present invention, in addition to the above objective(s), that insertion depth from port, and maneuverability of the vision system so provided is adjustable in a manner to work effectively in case of both pediatric as well as adult patients.

It is another objective of the present invention, in addition to the above objective(s), that operation of the vision system so provided is characterized in simple actuation, but high accuracy and precision.

It is another objective of the present invention, in addition to the above objective(s), that operation of the vision system so provided may be enabled via manual, semi-automated or fully-automated means.

It is another objective of the present invention, in addition to the above objective(s), that the vision system so provided is cost-effective to manufacture and capable of durable, long service life.

These and other objectives and their attainment will become apparent to the reader upon the detailed disclosures to follow.

### SUMMARY

In view of the foregoing wants of art, the present invention is directed towards the construction and implementation a purely novel self-illuminated and self-cleaning three-dimensional stereovision system for use in minimally invasive surgery. The present invention is directed to provide greater flexibility, wider view envelope at lower cost than comparable technologies currently available.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art will appreciate that they may readily use the conception and the specific embodiment disclosed as a basis for modifying or designing other structures for carrying out the same purposes of the present invention. Those skilled in the art will also realize that such equivalent constructions do not depart from the spirit and scope of the invention which shall be interpreted solely in its broadest form.

### BRIEF DESCRIPTION OF DRAWINGS

The present invention is explained herein under with reference to the following drawings, in which,

FIG. 1 is a schematic diagram to illustrate the implementation environment of the vision system for interventional surgery as provided in the present invention.

FIG. 2 is a schematic vertical cross-sectional view of the three-dimensional vision system for interventional surgery made according to the present invention.

FIG. 3 is a proximal-side perspective view of the holding sub-assembly as provided in the present invention.

FIG. 4 is a distal-side perspective view of the holding sub-assembly as provided in the present invention.

FIG. 5 is an enlarged schematic vertical cross-sectional view of the vision module and its constituent components as provided in the present invention.

FIG. 6 is a diagrammatic illustration to showcase the allowable field of movement of the visual module as provided in the present invention.

FIGS. 7(a to d) illustrate certain configurations/articulations of the visual module as provided in the present invention.

FIG. 8 is a schematic vertical cross-sectional view illustrating construction and assemblage of the rotary shaft as provided in the present invention.

FIG. 9 is a distal-side perspective view illustrating constituents of the vision module and configuration of actuating and connective elements received by said vision module as provided in the present invention.

FIG. 10 is a proximal-side perspective view illustrating constituents of the vision module and configuration of actuating and connective elements received by said vision module as provided in the present invention.

FIG. 11 is another proximal-side perspective view illustrating constituents of the vision module and configuration of actuating and connective elements received by said vision module as provided in the present invention.

FIG. 12 is a side-perspective view showcasing the deployment of actuating and connective elements at proximal end of insertion sleeve as provided in the present invention.

FIG. 13 is a side-perspective view showcasing assemblage of connectors and their linkages at mid-section of the insertion sleeve as provided in the present invention.

FIG. 14 is a distal side-perspective view showcasing deployment of various constituents, actuation and connective mechanisms received within the vision module as provided in the present invention.

FIG. 15 is a distal side view showcasing deployment of various constituents, actuation and connective mechanisms received within the vision module as provided in the present invention.

FIG. 16 is a distal side-perspective view of the distal end of the vision system for interventional surgery as provided in the present invention.

In above drawings, wherever possible, the same references and symbols have been used throughout to refer to the same or similar parts. References made to particular examples and implementations are for illustrative purposes, and are not intended to limit the scope of the invention or the claims. Numbering has been introduced to demarcate reference to specific components, such references being made in different sections of this specification. Not all components are marked in all drawings, but numbered in relation to context of the accompanying description.

Attention of the reader is now requested to the detailed description to follow which narrates a preferred embodiment of the present invention and such other ways in which principles of the invention may be employed without parting from the essence of the invention claimed herein.

### DEFINITIONS AND INTERPRETATIONS

Before undertaking the detailed description of the invention below, it may be advantageous to set forth definitions of certain words or phrases used throughout this patent document: the terms “include” and “comprise,” as well as derivatives thereof, mean inclusion without limitation; the term “or” is inclusive, meaning and/or; the phrases “associated with” and “associated therewith,” as well as derivatives thereof, may mean to include, be included within, interconnect, with, contain, be contained within, connect to or with, couple to or with, be communicable with, cooperate with, interleave, juxtapose, be proximate to, be bound to or with, have, have a property of, or the like; and the terms “proximal end” and “distal end” mean relative distance from the user/operator/surgeon while using the self-cleaning three-dimensional stereovision system for use in minimally invasive surgery proposed herein, and those of ordinary skill in the art will understand that such definitions apply in many, if not most, instances to prior as well as future uses of such defined words and phrases.

### DETAILED DESCRIPTION

Principally, general purpose of the present invention is to assess disabilities and shortcomings inherent to known systems comprising state of the art and develop new systems incorporating all available advantages of known art and none of its disadvantages. Accordingly, the disclosures herein are directed towards the construction and operation of a three-dimensional vision system for interventional surgery which is capable of efficiently meeting all major, if not all, objectives set out hereinbefore.

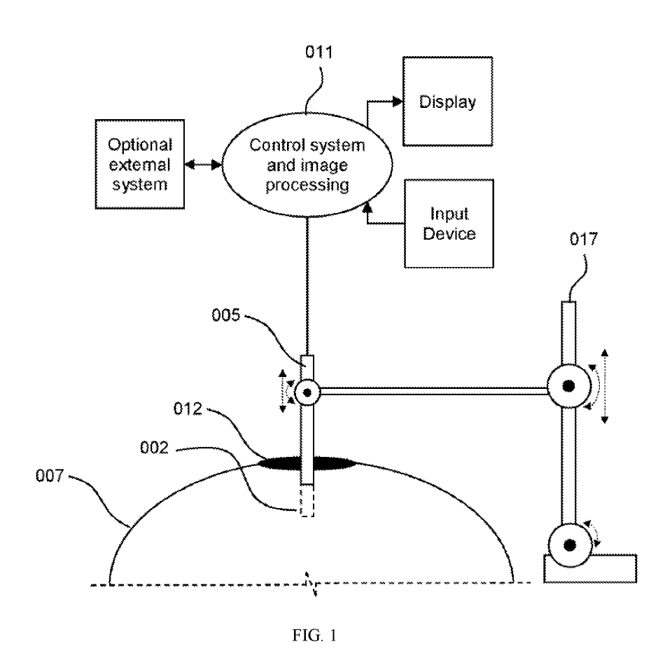

Construction of the vision system (001) for use in minimally invasive surgery proposed herein is intended to encompass various embodiments, among which a preferred and few alternative embodiments are explained below with general reference to the accompanying FIGS. 1 to 16 that illustrate generically the manner in which principles of the present invention may be employed.

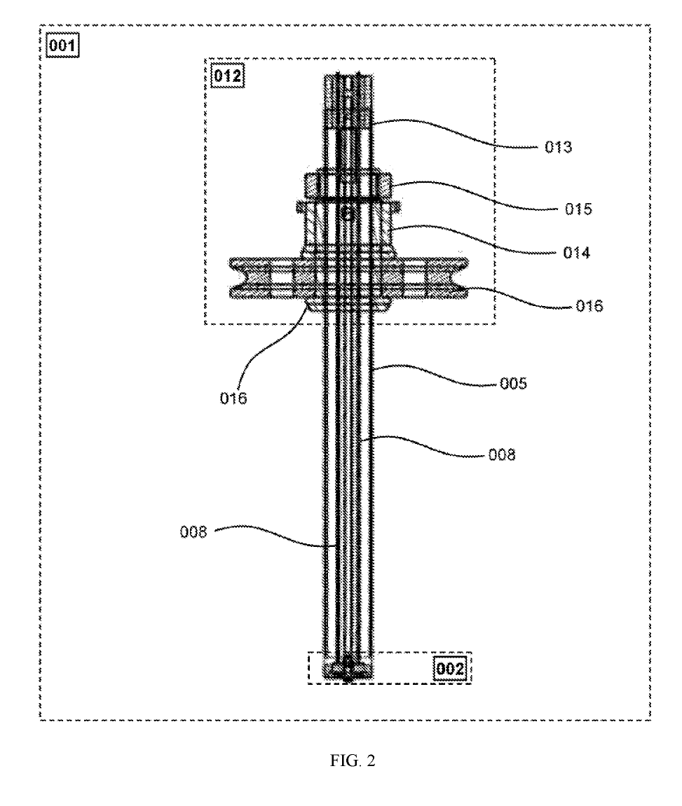

FIG. 1 is a schematic diagram to illustrate the implementation environment of the vision system (001) as provided in the present invention. Furthermore, the accompanying FIG. 2 is a schematic vertical cross-sectional view of the three-dimensional vision system (001) for interventional surgery as provided in the present invention. As seen in these illustrations, the three-dimensional vision system (001) for interventional surgery proposed herein, at outset, comprises a vision module (002) which is received at distal end of an insertion sleeve (005), and a sub-assembly (012) for positioning said sleeve (005) in working alignment relative to the CO₂-insufflated body cavity (007) of patient undergoing minimally invasive surgery. Furthermore, a segmented stand (017) having a heavy base and at least two independently articulating/locking arm segments is provided, in a preferred embodiment, for externally supporting and positioning the sleeve (005) while the system (001) is either idle or in use in the manner which will be particularly outlined in the narration to follow, described together with defining principles of construction, assemblage and deployment of further constituent components and further associations of the system (001).

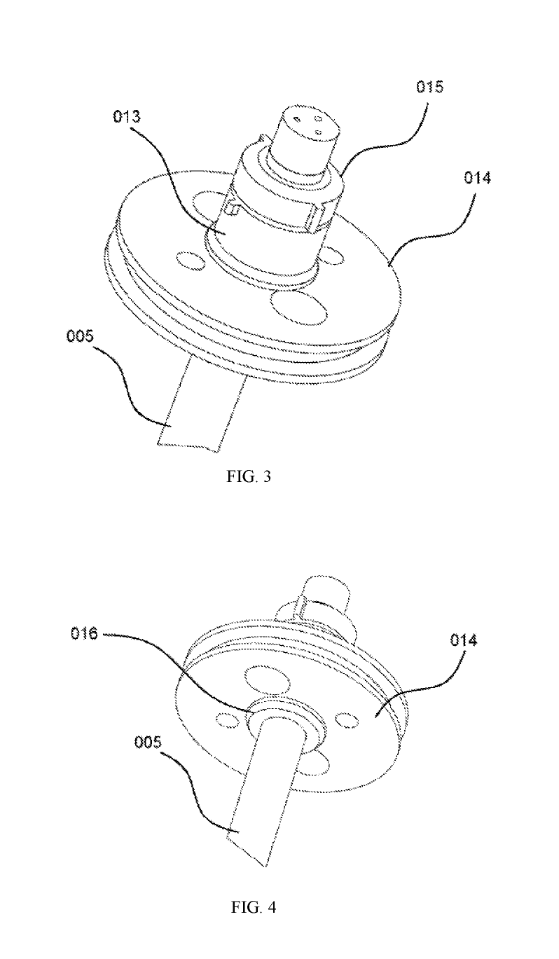

FIG. 3 and FIG. 4 represent proximal-side perspective view, and a distal-side perspective view, respectively of the holding sub-assembly (012) as provided in the present invention. Accordingly, placement of the sleeve (005) is aided, in one embodiment, in relation to the CO₂-insufflated body cavity of the patient undergoing minimally invasive surgery by help of a sub-assembly (012) which includes a proximal holding unit (013) a holding ring (014), sleeve locking ring (015) and a mounting unit (016) for secure operable insertion of the sleeve (005) along its longer axis. Construction and functions of these components (013, 014, 015, and 016) of the sub-assembly (012) are common to art and go by their nomenclature—hence not described in detail herein to avoid obscuring novelty of the present invention. It shall be evident to the reader, that in alternative embodiments hereof, the sleeve (005) is capable of being held in position at hand of a human operator, or by subassembly (012) or further alternatively, or in combination, with help of the stand (017).

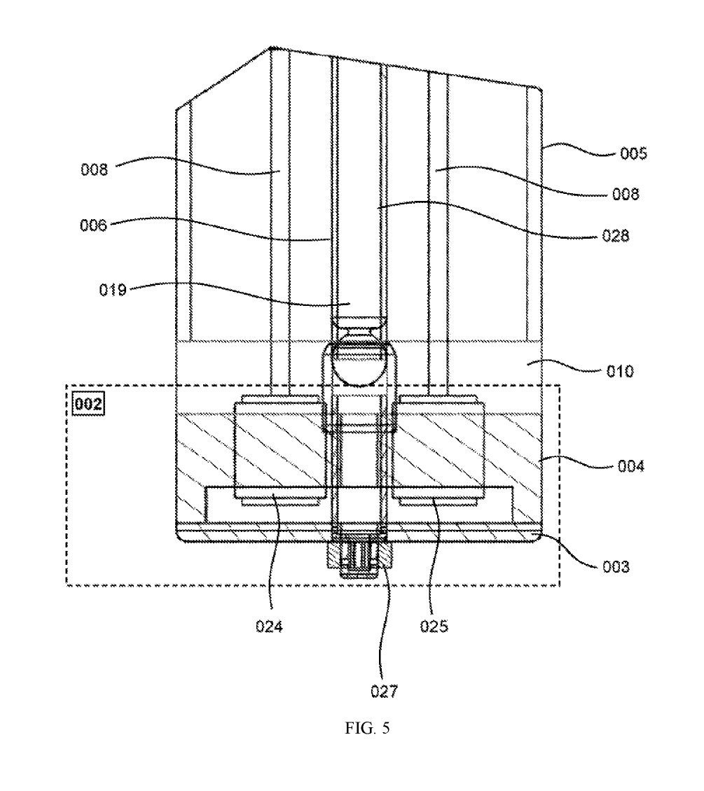

Functionally, the visual module (002) serves to host concatenated mechanisms for image capture, illumination, cleaning and dissipation of condensation in a manner that allows said means to be disposed freely into the insufflated body cavity of a patient undergoing minimally invasive surgery. The accompanying FIG. 5 is an enlarged schematic vertical cross-sectional view of the vision module (002) and its constituent components as provided in the present invention. As seen here, the vision module (002) constitutes in form, and function, as a detached extension of the sleeve (005) having preferably equal diameter relative to the sleeve (005). Essentially of opaque construction and cylindrical geometry, the module (002) characteristically is bounded by a transparent base (003) and a planar disc (004) at top, of which the base (003) serves as an observation window, and the planar disc (004) serves for attachment of sleeve (005) and actuating elements to be described later in this document. In another embodiment, construction of the module (002) may be achieved by arranging the circumferential lip of disc (004) to be extended perpendicularly to thereby form a cylindrical extension on which the transparent base (003) may be received thereby enclosing a lumen for hosting the aforesaid mechanisms for image capture, illumination, cleaning and dissipation of condensation. Cables (represented by common element 008) for data transfer and electrical power passed through bore of the sleeve (005) are provided for operation of the said means of image capture, illumination, cleaning and dissipation of condensation.

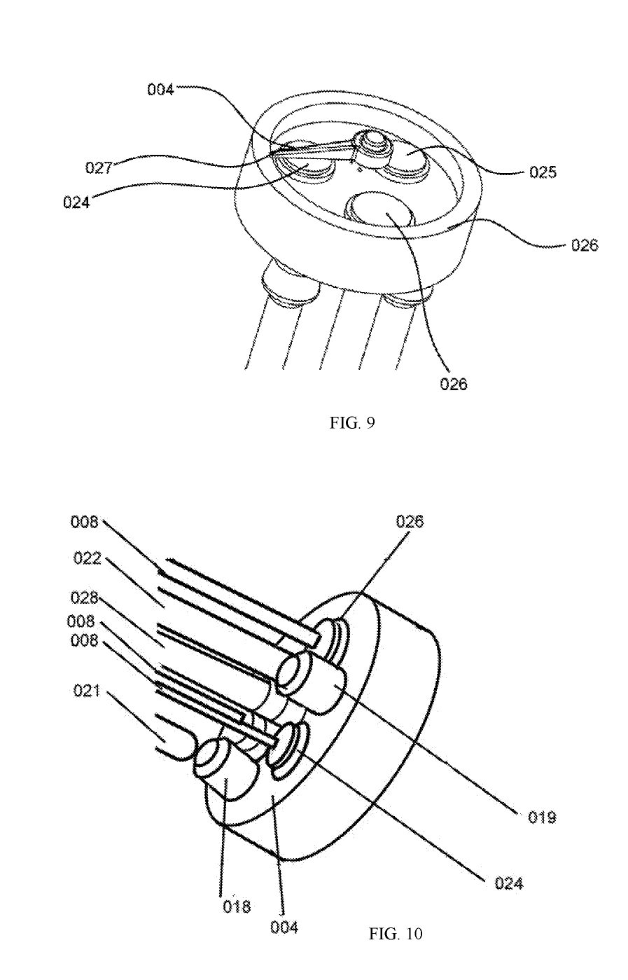

With continued reference to the accompanying FIG. 2, and furthermore the FIG. 9 and FIG. 10, the latter pair being a distal-side perspective view, and proximal-side perspective view respectively illustrating constituents of the vision module (002) and also configuration of actuating and connective elements received by said constituents as provided in the present invention, it can be seen that the means for illumination and image capture introduced hereinabove are mounted on the underbelly of the planar disc (004) so as to be oriented towards the base (003). This arrangement allows the means for image capture to obtain a forward-looking illuminated field of vision through the base (003) corresponding to the site of surgical intervention. As realized hereinabove, the mounting of said camera modules (024 and 025) or ability to alternatively mount a plurality thereof, on the same planar reference (004) ensures simultaneous movement of all camera modules involved, and thereby avoids further calibration required due to inaccuracies of different mounting references otherwise had in conventional state-of-art vision systems. Ability to mount multiple camera modules along with light source on the same reference thus ensures same relative light direction even after changing the field of view thereby avoiding further adjustment of the light source with respect to the camera module after changing the field of view.

The preferred embodiment of the present invention enlists a pair of stereoscopic camera modules (024 and 025) for image-capture, and a single light source (026) such as a light emitting diode module for illumination. It shall be understood that said modules for illumination and image capture may be alternatively sourced from common art devices designed for the purpose, for assimilation of their inherent features and advantages in further embodiments of the present invention. As said before, further embodiments of the present invention are intended wherein the image-capture means are interchangeable, or may be advantageously selected for deployment from among those available in common art therefore facilitating either of conventional still image, motion capture, two dimensional, and three-dimensional imaging their equivalents and their combinations as per requirement of the application scenario on hand.

With yet continued reference to the accompanying FIG. 2, and furthermore the FIG. 9 and FIG. 10, the latter pair being a distal-side perspective view, and proximal-side perspective view respectively illustrating constituents of the vision module (002) and also configuration of actuating and connective elements received by said constituents as provided in the present invention, it can be seen that the means for cleaning debris and/or fluids adhering to the base (003) is a rotary brush/wiper arrangement (027) that sits flush onto external surface of the base (003), and upon actuation via an external control unit (011), provides a circular sweeping action thereon to effectively clear the aforesaid debris and/or fluids adhering to the base (003), if any, during the surgical intervention underway. This arrangement ensures cleaning action and thus maintaining clear vision during use of the system (001).

With yet continued reference to the accompanying FIG. 2, and furthermore the FIG. 9 and FIG. 10, the latter pair being a distal-side perspective view, and proximal-side perspective view respectively illustrating constituents of the vision module (002) and also configuration of actuating and connective elements received by said constituents as provided in the present invention, it can be seen that the means for dissipation of condensation occurring in the lumen of vision module (002) are a supply of conditioned air that is supplied through a rotary hollow air tube (028)passing through bore of the sleeve (005) into the space within lumen of vision module (002). The supply of air is conditioned for temperature, humidity as per standard surgical procedures. As aforementioned, the rotary air tube (028) also transmits rotary motion/torque to the rotary brush/wiper arrangement (027) by means of manual rotation via suitable trigger or under action of a remotely connected rotary motor or servo. The tube (028) thereby effectively sucks or passes air to the gap between the base (003) and cameras (025 and 026) through an aperture (029) to thereby remove condensation occurring in the lumen of vision module (002). This arrangement avoids condensation on the camera modules (024 and 025), and light source (026) disposed within the lumen of module (002), thereby maintaining clear vision during use of the system (001).

Furthermore, FIGS. 7(a to d) illustrate certain configurations/articulations of the visual module as provided in the present invention according to which the planar disc (004), and thus the vision module (002) of which the disc (004) is a part, can be reciprocally oriented to face left, down, front, or right without moving the sleeve (005), or rotated infinitely about long axis of the sleeve (005), and furthermore elevated/descended towards the distal side to thereby allow a user to access a forward-looking, interference-free, rotatable, spherical view-envelope at the site of surgical intervention without change in orientation of the sleeve (005). The mechanics behind this motion, which constitute an important feature of the present invention are described in more detail in the disclosures to follow hereinunder.

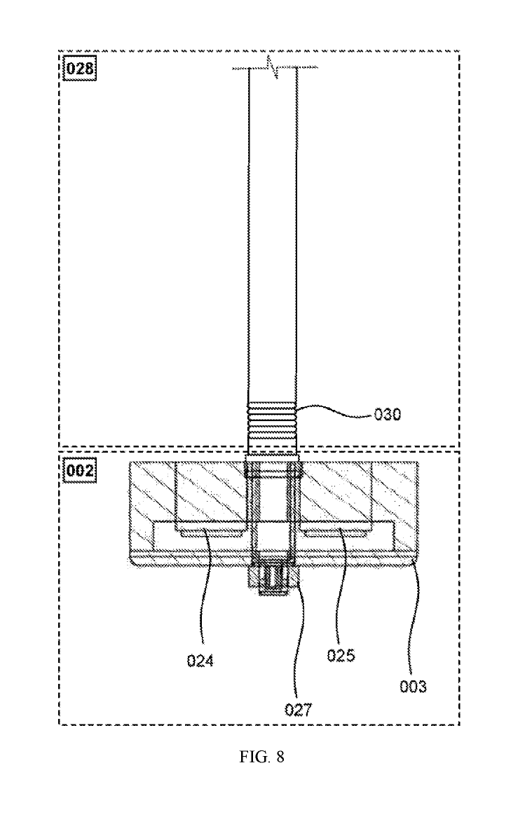

Referring specifically to FIG. 8, it can be seen that the rotary tube (028) comprises a flexible tube portion (030) towards its distal end. The distal end itself is attached to the planar disc (004) via suitable mechanism such as welding/adhesive or the like. As may be readily appreciated, the portion (030) allows the vision module (002) to be angled, in continuity, through positions depicted in FIGS. 7(a to d) and thus have infinite viewing planes at the site of surgical intervention. The incorporation of flexible tube portion (030) is responsible for provision of both the air supply for dissipation of condensation and also torque for rotary motion of the rotary brush/wiper arrangement (027).

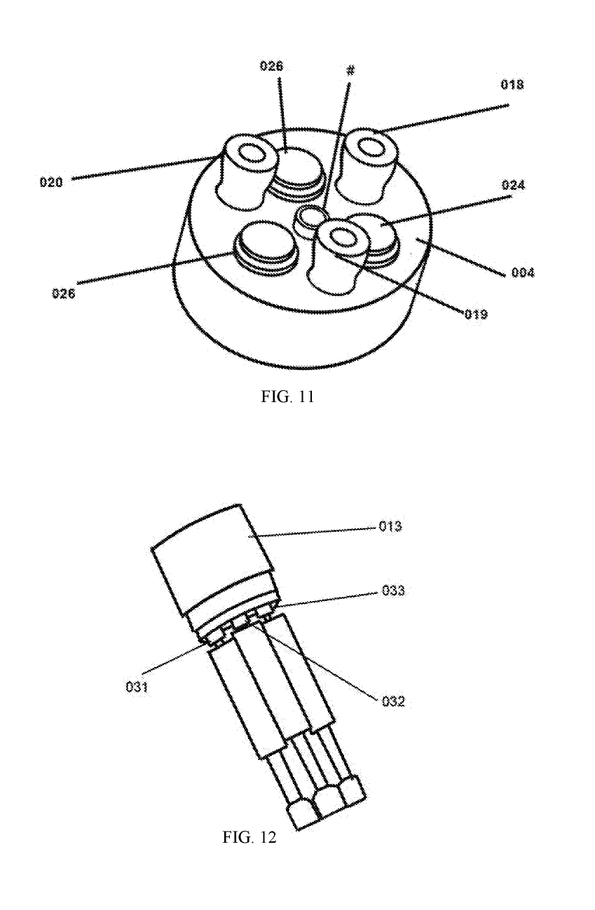

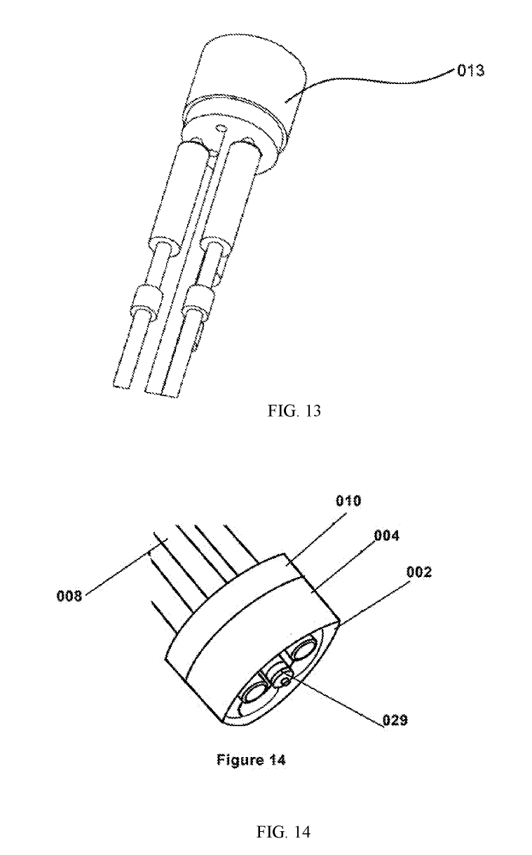

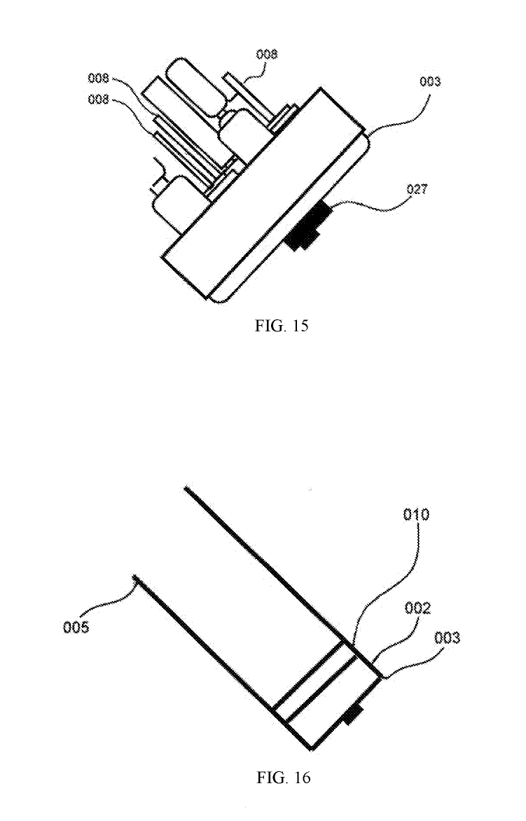

With continued reference to the accompanying FIG. 9 and FIG. 10, and particularly FIG. 11 which is a proximal-side perspective view illustrating constituents of the vision module (002) and configuration of actuating and connective elements received by said vision module (002); and FIG. 12 and FIG. 13, which are side-perspective views showcasing the deployment of actuating and connective elements at proximal end, and mid-section respectively of insertion sleeve as provided in the present invention, it can be seen that the mechanism which allows the vision module (002) to be angled, in continuity, through positions depicted in FIGS. 7(a to d) comprises linear displacement actuators being originated from within the sub-assembly (012) and received there subsequently at planar disc (004) after passing through bore of sleeve (005). This construction and operability may also be clearly referenced at FIG. 14 and FIG. 15, which are a distal side-perspective view and distal side view respectively showcasing deployment of various constituents, actuation and connective mechanisms received within the vision module as provided in the present invention.

Preferably, linear actuators (021, 022, and 023) ball-ended at their both proximal and distal ends which lead, via suitable connectors and their linkages/shaft extensions within bore of sleeve (005), ultimately into respectively mated ball-housings (18, 019, and 020) distally on the planar disc (004) and mated ball-housings (31, 32, and 33) at respective ends of linear actuator arm segments in proximal end of the sleeve (005) are used. The mated sets of ball-housings (18, 019, and 020) and (031, 032, and 033) help the linear actuators (021, 022, and 023), extended via suitable shafts and linkers to accommodate translational displacements/vector forces as the system (001) is guided through the positions depicted in FIGS. 7(a to d).

As seen in the accompanying FIG. 11, said sets of mated ball-housings (18, 019, and 020) form a triangle which defines a plane, and hence linear displacement of the vertices thus enabled along long axis of the sleeve (005) provides sufficient motion to manipulate the planar disc (004) to thereby attain a calibrated, user-defined rotation about long axis of sleeve (005), variable pitch of disc (004) and also elevation/descent by collapsing and elongation of the sealed flexible tube segment (030). The reader shall appreciate that this construction and assemblage makes the vision module (002) of which the disc (004) is a part motile, in a manner that can be reciprocally oriented to face left, down, back, front, or right without moving the sleeve (005), or rotated infinitely about long axis of the sleeve (005), and furthermore elevated/descended towards the distal side to thereby allow a user to access a forward-looking, interference-free, rotatable, spherical view-envelope at the site of surgical intervention without change in orientation of the sleeve (005).

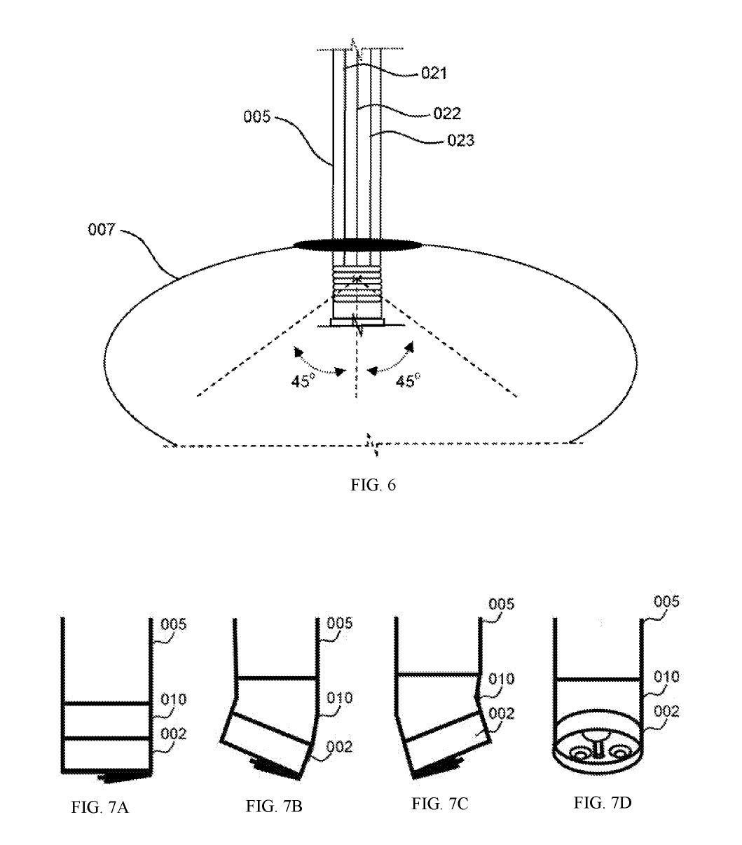

As a consequence of the operability provided hereinabove, the camera modules (024 and 025) and light source (026) that are mounted on said disc (004) thereby move along with motion of the disc (004) and therefore allow a user to control and access an illuminated three-dimensional stereo vision envelope at site of surgical intervention. A peculiar aspect of the present invention is thus realized that the module (002) is adapted for being manipulated in 360° space while being inserted within the CO₂-insufflated body cavity of a patient undergoing minimally invasive surgery. FIG. 6 is a diagrammatic illustration to explain the allowable field of movement of the visual module as provided in the present invention. Accordingly, the central axis of module (002) is allowed a conical maneuvering envelope defined by radial translation of said axis about an angle of 45° relative to long axis of the sleeve (005).

FIG. 16 is a distal side-perspective view of the distal end of the finalized vision system for interventional surgery as provided in the present invention. A flexible cylindrical sleeve (010) is introduced in-between said sleeve (002) and module (002) which maintains enclosure between the respective lumens of sleeve (005) and module (002) at all times thus sealing out the external environment.

As per the foregoing narration, an able three dimensional vision system for interventional surgery is thus provided with improved functionality, durability and long service life than any of its closest peers in state-of-art. Materials of construction, though not materially defining the present invention, may be advantageously selected from state-of-art biocompatible materials either presently prevalent, or as may be developed in the future, in the technical field of the present invention.

From the principles of implementation reflected hereinabove, it would be evident to the reader, that entry of said sleeve (005) into body cavity (007) of the patient is typically arranged via a surgical port, or a natural body orifice of the patient to thereby minimize necessity of larger incisions and particularly the complications and trauma associated with such larger incisions.

Consequentially, this capability of the system (001) to avoid larger cuts and ensuring lesser trauma to patient also promises less post-operative pain and faster recovery of the patient post-surgery. That all the objectives set out in the foregoing part of this document have been effectively met shall be abundantly clear to the reader, in advantage of the disclosures provided hereinabove.

As will be realized further, the present invention is capable of various other embodiments and that its several components and related details are capable of various alterations, substitutions, variations, enhancements, nuances, gradations, lesser forms, alterations, revisions, improvements and knock-offs, all without departing from the basic concept of the present invention. Accordingly, the foregoing description will be regarded as illustrative in nature and not as restrictive in any form whatsoever. Without exception, these are intended to come within ambit of the present invention, which is limited only by the appended claims.

We claim:

1. A vision system (001) for use in minimally invasive surgery, comprising:
a) A vision module (002) being a hollow cylinder bounded between a transparent base (003) and a planar disc (004) at top, being capable of hosting at least one each among means for image capture, illumination, cleaning and dissipation of condensation;
b) A hollow, insertion sleeve (005) of outer diameter equal to that of the module (002) and having a central shaft (028) passing through its bore, being capable of receiving the vision module (002) at a distal end and thereafter extending a through a surgical port, and alternatively a body orifice, into a insufflated body cavity (007) of a patient undergoing minimally invasive surgery;
c) supporting means for securely positioning the sleeve (005) when in idle state, and alternatively extending into the insufflated body cavity (007) of a patient undergoing minimally invasive surgery;
d) cables (008) for conveying data, and alternatively electrical power, passing through a bore of the insertion sleeve (005) and leading to the said means for image capture, illumination, cleaning and dissipation of condensation; and e) external means for control and display in connection with the cables (008) for allowing a user to operate the system (001),
Characterized in that the vision module (002) is arranged for allowing a user to access a 360° rotatable, spherical, dome-shaped, forward-looking, illuminated view-envelope having infinite planes of vision at a site of surgical intervention without movement of the sleeve (005), by inclusion of adaptations including: i) the means for image capture and illumination are mounted on a distal-side face of the planar disc (004) in a manner looking onto the site of surgical intervention through the transparent base (003);
ii) the planar disc (004), and thus the vision module (002) borne thereon, is arranged to rotate infinitely, extend, descend, and circumscribe a conical maneuvering envelope around a stationary central long axis of the central shaft (028) in a manner identified in being independent of the sleeve (005), and sealing out an external environment; and
iii) means for cleaning and dissipation of condensation are arranged on the central shaft (028) in a manner identified in being independent of a maneuvering envelope of the planar disc (004).

2. The vision system (001) for use in minimally invasive surgery as claimed in claim 1, wherein the planar disc (004) is arranged to rotate infinitely, extend, descend, and circumscribe a conical maneuvering envelope, at an angle of up to 45° around the stationary central long axis of the central shaft (028) in a manner identified in being independent of the sleeve (005), and sealing out the external environment by incorporation of a concerted synergistic mechanism including:
a) an airtight, collapsible, and flexible tube segment (030) interposed co-axially in the shaft (028) at a position immediately before the shaft (028) passes through a centre of the planar disc (004) to enable the planar disc (004), and thus the vision module (002) borne thereon, to rotate infinitely around the central long axis of the central shaft (028) and additionally extend, descend according to flexibility limits of a tube segment (030);
b) three arm shafts (021, 022, and 023), each of which is ball-ended at both ends and capable of being attached in a first pivot association, to linear displacement actuators at a proximal end of the sleeve (005) via set of ball-housings (031, 032, and 033), and subsequently in a second pivot association to a proximal face of the planar disc (004) via a set of ball-housings (018, 019, and 020), to result in that a motion of arm shafts (021, 022, and 023) in a triangular configuration so reached causes variation in a plane, and thus pitch of the planar disc (004) about the central long axis of the central shaft (028); and
c) a flexible cylindrical sleeve (010) of outer diameter equaling the sleeve (005) introduced between a sleeve (002) and the module (002) which maintains enclosure between the respective lumens of the sleeve (005) and the module (002) at all times thus allowing motion of the vision module (002) but sealing out the external environment.

3. The vision system (001) for use in minimally invasive surgery as claimed in claim 1, wherein the external means for control and display in connection with the cables (008) for allowing the user to operate the system (001) are an interactively linked couple comprising:
a) An external display unit, being a monitor in particular, for allowing the user to view the site of surgical intervention inside the insufflated body cavity (007) of the patient undergoing minimally invasive surgery; and
b) an external control unit (011) including triggers selected among a joystick, turning knobs, buttons, trigger levers, toggle levers, switches, their equivalents and their combinations for allowing the user to alternatively orient the vision module (002) for attaining a suitable field of view; to zoom in, and out, of the field of view selected; and actuate, as needed, among the means for cleaning and dissipation of condensation in the event debris and condensate respectively adhering onto the transparent base (003) of the vision module (002).

4. The vision system (001) for use in minimally invasive surgery as claimed in claim 1, wherein the supporting means for secure positioning of the sleeve (005) and thereby avoiding need for an additional human operator are selected singly, and alternatively in combination, among:
a) a sub-assembly (012) including a proximal holding unit (013), a holding ring (014), a sleeve locking ring (015) and a mounting unit (016) being positioned atop the surgical port, and alternatively the body orifice, into the insufflated body cavity (007) of the patient undergoing minimally invasive surgery; and
b) a stand (017) having a heavy base and at least two articulating arm segments the distal end of which is equipped with a gripper mount for securely receiving the sleeve (005).

5. The vision system (001) for use in minimally invasive surgery as claimed in claim 1, wherein the sleeve (005) is required to be inserted at a minimal depth into the insufflated body cavity of the patient undergoing minimally invasive surgery substantially in a manner that leaves a greater part of a proximal length free outside a body of the patient, and the inserted distal end being oriented in a direction generally facing the site of surgical intervention to thereby ensure compatibility with both pediatric and adult patients and minimal interference with other surgical equipment participating in the surgical intervention.

6. The vision system (001) for use in minimally invasive surgery as claimed in claim 1, wherein the means for image capture capable of recording the field of view corresponding to the site of surgical intervention in response to actuation via the external control unit (011) are a pair of stereoscopic camera modules (024, and 025).

7. The vision system (001) for use in minimally invasive surgery as claimed in claim 1, wherein means for illumination capable of illuminating the field of view corresponding to the site of surgical intervention in response to actuation via the external control unit (011) are selected among a single light emitting diode module (026) hosted on a distal face of the planar disc (004), and alternatively an illumination ring circumscribing the transparent base (003).

8. The vision system (001) for use in minimally invasive surgery as claimed in claim 1, wherein the means for cleaning capable of clearing fluids and debris adhering onto a distal face of transparent base (003) are a single radial wiper (027) arranged to perform a circular sweeping motion upon the distal face of transparent base (003) upon torque provided by the central shaft (028) under actuation via the external control unit (011).

9. The vision system (001) for use in minimally invasive surgery as claimed in claim 1, wherein the means for dissipation of condensation capable of clearing condensate accumulated within a enclosed space between the planar disc (004) and the base (003) of the vision module (002) in response to actuation via the control unit (011) are a supply of conditioned air conveyed through the central shaft (028) which opens via an aperture (029) arranged within the space bounded by the planar disc (004) and the transparent base (003) to thereby avoid condensation building up on the means of image capture and illumination.

10. The vision system (001) for use in minimally invasive surgery as claimed in claim 6, wherein the means for image capture, illumination, cleaning and dissipation of condensation respectively mentioned are further alternatively selected among conventional systems known for said purposes, their equivalents and their combinations suitable for use in minimally invasive surgery.

11. The vision system (001) for use in minimally invasive surgery as claimed in claim 4, wherein the means for illumination capable of illuminating the field of view corresponding to the site of surgical intervention in response to actuation via the external control unit (011) are selected among a single light emitting diode module (026) hosted on the distal face of the planar disc (004), and alternatively an illumination ring circumscribing the transparent base (003).

12. The vision system (001) for use in minimally invasive surgery as claimed in claim 4, wherein the means for cleaning capable of clearing fluids and debris adhering onto the distal face of transparent base (003) are a single radial wiper (027) arranged to perform a circular sweeping motion upon said distal face of transparent base (003) upon torque provided by the central shaft (028) under actuation via the external control unit (011).

13. The vision system (001) for use in minimally invasive surgery as claimed in claim 4, wherein the means for dissipation of condensation capable of clearing condensate accumulated within the enclosed space between the planar disc (004) and the base (003) of the vision module (002) in response to actuation via the control unit (011) are a supply of conditioned air conveyed through the central shaft (028) which opens via an aperture (029) arranged within a space bounded by the planar disc (004) and the transparent base (003) to thereby avoid condensation building up on the means of image capture and illumination.

14. The vision system (001) for use in minimally invasive surgery as claimed in claim 7, wherein the means for image capture, illumination, cleaning and dissipation of condensation respectively mentioned are further alternatively selected among conventional systems known for said purposes, their equivalents and their combinations suitable for use in minimally invasive surgery.

15. The vision system (001) for use in minimally invasive surgery as claimed in claim 8, wherein the means for image capture, illumination, cleaning and dissipation of condensation respectively mentioned are further alternatively selected among conventional systems known for said purposes, their equivalents and their combinations suitable for use in minimally invasive surgery.

16. The vision system (001) for use in minimally invasive surgery as claimed in claim 9, wherein the means for image capture, illumination, cleaning and dissipation of condensation respectively mentioned are further alternatively selected among conventional systems known for said purposes, their equivalents and their combinations suitable for use in minimally invasive surgery.
